(19) United States
(12) Patent Application Publication
(10) Pub. No.: US 2016/0296201 A1
(43) Pub. Date: Oct. 13, 2016

(54) Imaging and measuring system of vocal cord vibration based on plane wave ultrasonography, and method thereof

(71) Applicant: XI'AN JIAOTONG UNIVERSITY, Xi'an, Shaanxi (CN)

(72) Inventors: Mingxi Wan, Xi'an, Shaanxi (CN); Shanshan Tang, Xi'an, Shaanxi (CN); Bowen Jing, Xi'an, Shaanxi (CN); Supin Wang, Xi'an, Shaanxi (CN)

(73) Assignee: XI'AN JIAOTONG UNIVERSITY, Xi'an, Shaanxi (CN)

(21) Appl. No.: 15/025,564

(22) PCT Filed: Dec. 19, 2014
(86) PCT No.: PCT/CN2014/094449

§ 371 Date: Mar. 29, 2016

(30) Foreign Application Priority Data
Oct. 30, 2014 (CN) 201410605785.5

(51) Int. Cl. A61B 8/08; A61B 8/00; A61B 8/14

(52) U.S. Cl. CPC A61B 8/08; A61B 8/14; A61B 8/4483; A61B 8/5223

(57) ABSTRACT

An imaging and measuring system of vocal cord vibration based on plane wave ultrasonography, includes: a digital ultrasonography system, a data acquisition unit, and a computer; wherein the digital ultrasonography system comprises an ultrasound linear array transducer and a host; wherein the ultrasound linear array transducer is controlled by the host for sending an ultrasound plane wave and receiving echo; the echo is sent back to data acquisition unit; wherein the data acquisition unit converts the echo into digital signal and then sends it to the computer; wherein the computer provides beamforming, envelope detection, and dynamic range compression of the digital signal received, for obtaining a laryngeal tissue structure image. The present invention provides high-speed imaging of the vocal cord vibration with temproal and spatial synchronization, and quantitatively extracting information of biomechanical parameters as well as vibrational phase changes.

### CROSS REFERENCE OF RELATED APPLICATION

This is a U.S. National Stage under 35 U.S.C 371 of the International Application PCT/CN2014/094449, filed Dec. 19, 2014, which claims priority under 35 U.S.C. 119(a-d) to CN 201410605785.5, filed Oct. 30, 2014.

### BACKGROUND OF THE PRESENT INVENTION

1. Field of Invention

The present invention relates to a field of biomedicine information detection, and more particularly to high-speed vibration imaging which provides time-space synchronization of vocal cords, as well as a system for quantitatively extracting time-space vibration properties of the vocal cords and a method thereof.

2. Description of Related Arts

High-speed, complex and multi-dimensional vibration of human vocal cords generates a voice source. The vocal cords are small organs with highest vibration speeds of a human body, but are also most vulnerable organs. However, conventionally, researches of problems such as how the vocal cords in the body regulates biomechanical properties thereof to change a sound mode, and how lesions change the biomechanical properties of the vocal cords to caused pathological voice, are still in an infancy.

According to anatomical structures and hierarchical models, the vocal cords are divided into two layers: a body layer and a coating layer. The vibration of the vocal cords is in fact a combined vibration effect of the two layers with different biomechanical properties. Conventionally, researches on the vibration of the vocal cords are mostly focused on the coating layer, due to easy observation and record thereof through a throat endoscope. However, optical imaging technologies for larynx and vocal cords, comprising strobe dynamic laryngoscope and high-speed photography laryngoscope, are all not able to provide vibration imaging of internal organizational structures below surfaces of the vocal cords. Furthermore, due to invasion of the endoscope of the optical device, a patient is not able to pronounce in a natural voice.

Electrical glottograph (EGG) as a research method that reflects periodical changes of a contacting area of the vocal cords during pronouncing, has been widely used in clinical examinations and scientific researches of the vocal cords. Feature points extracted from the EGG and differential electrical glottograph (DEGG) are corresponding to physiological action time points which have special meanings in vocal cord vibration. In addition, features of the EGG, such as high time resolution and being easy to extract and record, make it possible to identify phase changes of vocal cord movements. However, an EGG signal is a one-dimensional comprehensive signal, and describes an overall situation of an entire vocal cord contacting area, because the EGG signal is a cumulative measurement of all point contacts of the vocal cords along a glottis direction. Therefore, the EGG is unable to describe quantify vibration properties of a certain tissue region of the vocal cords.

Compared with the above technologies, an advantage of medical ultrasonography is being non-invasive, which is able to provide imaging of tissue structures under the surface of the vocal cords while a testee is naturally pronouncing. However, a conventional ultrasonography technique uses a line-by-line scan mode, which divides an image into a number of scan lines, while data of each scan line are obtained at different moments, which leads to certain time differences between different points of the image during sampling. When it comes to the high-speed vibration of the vocal cords, the time differences cannot be ignored. In this case, the image will be blurred because of the high-speed vibration of the vocal cords, resulting in uncertain measurement of vibration velocity and displacement of the vocal cords. In addition, because of a low imaging frame rate (<1000 Hz) of the conventional ultrasonography technique, requirements of vocal cord vibration under unstable situations are unable to be satisfied.

Ultrasound glottograph (UGG) is another non-invasive method for observing dynamic processes of the vocal cords. However, the UGG conventionally reported adopts a single array element ultrasound transducer. A transmitting beam of the single array element ultrasound transducer has a strong directivity, which is not able to detect an overall structure and positions of the vocal cords. Without image guide, the single array element ultrasound transducer is easy to lose information during detection. However, a linear array transducer, which is capable of imaging of the vocal cord vibration within a range of an entire vocal cord length, has a limited application. Besides a low imaging frame rate of linear scan, another main reason is that under a limited linear scanning speed of ultrasound linear scan, the tissue structures at different position of a B-ultrasound image are not simultaneously sampled. Since the UGG reflects phase information of the vocal cord vibration, asynchronous problem is unacceptable.

So, high-speed imaging of the vocal cord vibration with time and space synchronization, and quantitatively extracting information of biomechanical parameters as well as certain phase changes are still major problems in the field.

### SUMMARY OF THE PRESENT INVENTION

An object of the present invention is to provide an imaging and measuring system of vocal cord vibration based on plane wave ultrasonography and a method thereof, so as to overcome problems and limits of the above conventional technologies in researches of vocal cords; wherein the present invention uses plane wave ultrasonography (PWU for short) for imaging of vocal cord vibration and quantization of vocal cord vibration properties.

Accordingly, in order to accomplish the above object, the present invention provides:

an imaging and measuring system of vocal cord vibration based on plane wave ultrasonography, comprising: a digital ultrasonography system, a data acquisition unit, and a computer; wherein the digital ultrasonography system comprises an ultrasound linear array transducer and a host; wherein the ultrasound linear array transducer is controlled by the host for sending an ultrasound plane wave and receiving an echo; the echo is sent back to the host; wherein the host controls the ultrasound linear array transducer for sending the ultrasound plane wave, and sends back the echo to the data acquisition unit; wherein the data acquisition unit converts a received echo signal into a digital signal and then sends to the computer; wherein the computer provides beam formation of echo data, radio frequency signal envelope detection, and dynamic range compression of the digital signal received, for obtaining a laryngeal tissue structure image.

Preferably, the ultrasound linear array transducer is placed on a neck surface of a testee along a coronal section, or along a cross section.

Preferably, an imaging frame rate of the digital ultrasonography system is 5000 frames per second, a center frequency of the ultrasound linear array transducer is 7.2 MHz.

Preferably, the ultrasound linear array transducer is placed on a neck surface of a testee along a coronal section; and the computer extracts a vibration displacement of a vocal cord body layer, a false vocal cord vibration displacement, and an initial vocal cord displacement from the laryngeal tissue structure image with a 2-dimensional motion estimation algorithm based on ultrasound radio frequency echo data.

Preferably, the ultrasound linear array transducer is placed on a neck surface of a testee along a cross section; and the computer extracts feature points and phase parameters of vocal cord vibration from the laryngeal tissue structure image.

The present invention also provides a method for imaging of vocal cord vibration based on plane wave ultrasonography, comprising steps of: placing ultrasound linear array transducers on a skin surface on a side of a testee neck, which is corresponding to a glottis position, along a coronal section and/or a cross section; sending an ultrasound plane wave to a laryngeal portion by the ultrasound linear array transducers, and receiving an echo, then sending the echo to a data acquisition unit; converting a received echo signal into a digital signal by the data acquisition unit and then sending to a computer; providing beam formation of echo data, radio frequency signal envelope detection, and dynamic range compression of the digital signal received, by the computer, for obtaining a laryngeal tissue structure image.

The present invention also provided a method for imaging of vocal cord vibration based on plane wave ultrasonography, comprising steps of: collecting a laryngeal tissue structure image by a computer, extracting a vibration displacement of a vocal cord body layer, a false vocal cord vibration displacement, and an initial vocal cord displacement from a laryngeal tissue structure image with a 2-dimensional motion estimation algorithm based on ultrasound radio frequency echo data.

Preferably, the laryngeal tissue structure image is obtained by: placing an ultrasound linear array transducer on a skin surface on a side of a testee neck, which is corresponding to a glottis position, along a coronal section; sending an ultrasound plane wave to a laryngeal portion by the ultrasound linear array transducers, and receiving an echo, then sending the echo to a data acquisition unit; converting a received echo signal into a digital signal by the data acquisition unit and then sending to the computer; providing beam formation of echo data, radio frequency signal envelope detection, and dynamic range compression of the digital signal received, by the computer.

The present invention also provides a method for imaging of vocal cord vibration based on plane wave ultrasonography, comprising steps of: collecting an ultrasound glottograph (UGG for short), which is sampled by an ultrasound linear array transducer, by a computer; judging positions of an anterior commissure and an arytenoid cartilage, and connecting the positions in an ultrasound image with a line, wherein the line coincides with a central line of a glottis; then selecting a rectangle as region of interest (ROI for short), wherein the line which coincides with the central line of the glottis is a symmetry axis of the rectangle; then evenly dividing the ROI into a plurality of segments along a vocal cord length direction; extracting gray values of all pixels in each of the segments of the ROI, and calculating a time-varying UGG of each of the segments of the ROI according to an equation (3):

$$U G G ( t ) = norm ( - \frac{1}{N} {\sum }_{i , j} {P}_{i , j} ( t ) ) \qquad ( 3 )$$

wherein, UGG(t) is the time-varying UGG, P_{i,j}(t) is a gray value of a pixel point (i,j) in the ROI at a time t; N is a quantity of all the pixels in the ROI; norm represents a normalizing operation; wherein the ROI is divided into M segments, and for each of the segments of the ROI, a corresponding UGG is extracted;

finding regular intercourse curves with large and small amplitudes from the corresponding UGG extracted; combining the curves for obtaining an overall UGG of vocal cord vibration; processing the UGG with differential for obtain a differential ultrasound glottograph (DUGG for short), calculating a D2UGG according to an equation (5):

D2UGG=DUGG(n)|DUGG(n)| (5)

according to a peak detection algorithm, obtaining an echo intensity weakest point in a glottis close phase and an echo intensity weakest point in a glottis open phase of the overall UGG; wherein a maximum glottis open time point is a second over-zero point after the echo intensity weakest point in the glottis open phase of the overall UGG; a glottis close time point is a first positive peak before an echo intensity weakest point in a glottis close phase of the D2UGG; and a glottis open time point is a negative peak of the D2UGG;

calculating a glottis closure quotient (CQ for short) according to an equation (7):

$$C Q = \frac{Loc ( F ) - Loc ( G )}{{T}_{egg}} \qquad ( 7 )$$

wherein Loc(F) represents a time position of the negative peak of the D2UGG, Loc(G) represents a time position of a position peak of the D2UGG, and T_{egg} represents a vibration period length.

Preferably, the UGG is obtained by: placing the ultrasound linear array transducer on a skin surface on a side of a testee neck, which is corresponding to a glottis position, along a cross section; sending an ultrasound plane wave to a laryngeal portion by the ultrasound linear array transducers, and receiving an echo, then sending the echo to a data acquisition unit; converting a received echo signal into a digital signal by the data acquisition unit and then sending to the computer, for obtaining a time-varying echo intensity curve.

Compared with the conventional technologies, advantages of the present invention are as follows.

1) Vocal Cord Tissue Vibration Imaging Method Based on the PWU with EGG Synchronization

A non-invasive imaging and detecting system is established, wherein the PWU is able to provide space-synchronized imaging of the vocal cord vibration, while a very high time resolution is achieved, which satisfies quantitative imaging requirements of the vocal cord vibration.

Firstly, in order to overcome motion blur problems of the conventional ultrasonography, the present invention abandons a linear scanning method adopted in conventional ultrasonography technologies. Instead, the present invention adopts a plane wave sending method. By sending a plane ultrasound wave, a large area of a laryngeal portion is covered, so as to obtain a laryngeal tissue structure image in an entire plane. In a direction perpendicular to a sound beam, each section of the image is simultaneously sampled, which greatly avoids sampling time differences between scanning lines of the conventional ultrasonography technologies, so as to greatly improve the motion blur problems of the vocal cord tissue vibration imaging. The imaging frame rate of the method is up to 7000 frames per second, which is far greater than a vocal cord vibration frequency. Therefore, the method is suitable for researches of aperiodic irregular vibration of the vocal cords under a non-stable pronouncing condition.

During imaging, the ultrasound linear array transducer is place on the side of the testee neck, which is corresponding to the glottis position, for judging laryngeal tissue structures of the vocal cords and false vocal cords. An operator is able to obtain tissue structure images at a vocal cord coronal section and a horizontal plane by adjusting positions and angles of the ultrasound linear array transducer. When the testee tells a vowel, PWU technologies are used for sampling original echo data of vocal cord high-speed vibration. After the beam formation, the radio frequency signal envelope detection, and the dynamic range compression, the echo data is converted into the laryngeal tissue structure image.

2) Measuring and Detecting Method of Up-Down Tissue Vibration of the Vocal Cords and the Glottis with a 2-Dimensional Motion Estimation Algorithm Based on Plane Wave Radio Frequency Data

The original echo data is treated with the 2-dimensional motion estimation algorithm based on the plane wave radio frequency data, for obtaining vibration velocity vectors and displacements of vocal cord tissues in the coronal section. The vocal cord vibration causes delay between data of adjacent frames. By estimating the delay, displacement vectors of the tissue during a sampling interval is able to be obtained. By dividing the displacements with the sampling interval, vibration velocities of the vocal cord tissues are obtained. Compared with other motion estimation algorithms based on radio frequency data treated by the beam formation, the algorithm of the present invention has a higher lateral displacement resolution, so as to detect tissue vibration with smaller amplitudes. On a basis that tissue vibration velocities and displacements are obtained, vocal cord tissue vibration frequencies and amplitudes are further obtained.

The method is capable of not only quasi-periodic vibration imaging and measuring under stable pronouncing conditions, but also aperiodic irregular vibration imaging and measuring under non-stable pronouncing conditions. Meanwhile, the method has a wide imaging view field, so as to measure glottis up-down vibration and vocal cord surrounding tissue vibration, such as false vocal cord vibration.

3) Segmented UGG Method Based on the PWU Technologies

A method for extracting the UGG based on the PWU is disclosed, comprising steps of firstly judging the positions of the anterior commissure and the arytenoid cartilage in the ultrasound image of the vocal cord cross section, and connecting the positions for determining the central line of the glottis; then selecting the rectangle as the ROI with the central line as the symmetry axis of the rectangle; then dividing the ROI into the segments according to requirements; and calculating an overall time-varying UGG of the entire vocal cords along the vocal cord length direction, and segmented UGG of certain portions of the vocal cords.

4) Extraction of the Feature Points and Feature Parameters of the Segmented Ultrasound UGG

According to the peak detection algorithm and a zero-crossing detection algorithm, the feature points are available from the UGG, comprising the maximum glottis open time, the glottis close time and the glottis open time. The glottis closure quotient is an important time phase parameter, representing a ratio of a vocal cord close period and an entire vocal cord vibration period. Conventionally, the glottis close time is determined by extracting a positive peak and a negative peak of the DEGG, whose reliability is lowered due to the insufficient negative peak of the DEGG, resulting in low accuracy of the glottis closure quotient. According to the present invention, the negative peak obtained by the UGG method is significant, which is highly reliable during extraction. Therefore, in the present invention, the EGG method is combined with the UGG method to extract the glottis closure quotient, wherein by the glottis closure quotient is calculated by extracting the positive peak of the DEGG and the negative peak of the DUGG, thereby improving the accuracy of the glottis closure quotient, which is the important time phase parameter of the vocal cord vibration.

According to the present invention, the imaging and detecting method is non-invasive, which causes minimal interference during pronouncing, so as to ensure that the testee is able to naturally pronounce and dynamically pronounce.

The plane wave imaging technologies are able to eliminate space asynchronism of the vocal cord vibration imaging, while point synchronism of the EGG eliminates time randomness caused by ultrasound during sampling the vocal cord vibration. Thus, the present invention is able to provide the time-space synchronization of the vocal cord vibration detection.

The present invention is capable of comprehensively quantitative extraction of motion information of the vocal cords and surrounding tissues thereof, feature point information, and feature parameter information.

### BRIEF DESCRIPTION OF THE DRAWINGS

FIG. 1 is a flow chart of an imaging and detecting method of vocal cord vibration based on plane wave ultrasonography.

FIG. 2 is a sketch view of an ultrasonography transducer placed on a coronal section.

FIG. 3(a) is a sketch view of 2-dimensional motion estimation algorithm based on ultrasound radio frequency echo data.

FIG. 3(b) is a laryngeal tissue structure image converted from echo data.

FIG. 3(c) is vibration displacement curves of vocal cord tissues.

FIG. 4(a) is a sketch view of an imaging and detecting system of the vocal cord vibration based on the plane wave ultrasonography.

FIG. 4(b) is a sketch view of relative positions of the ultrasound transducer, vocal cords and surrounding tissues thereof.

FIG. 4(c) is a sketch view of positions of the ultrasound transducer and electrodes.

FIG. 5(a) is a sketch view of a region of interest (comprising positions of an anterior commissure and an arytenoid cartilage).

FIG. 5(b) is a sketch view of division of the ROI (comprising the positions of the anterior commissure and the arytenoid cartilage).

FIG. 6 is a segmented UGG and a synchronized EGG thereof.

FIG. 7 is an overall UGG and a synchronized EGG thereof.

### DETAILED DESCRIPTION OF THE PREFERRED EMBODIMENT

Referring to the drawings and a preferred embodiment, the present invention is further illustrated.

Referring to FIGS. 1-7, an imaging and measuring system of vocal cord vibration based on plane wave ultrasonography, comprising: a digital ultrasonography system, a data acquisition unit, and a computer.

The digital ultrasonography system comprises an ultrasound linear array transducer and a host; wherein the ultrasound linear array transducer is controlled by the host for sending an ultrasound plane wave and receiving an echo; the echo is sent back to the host; wherein the host sends back the echo to the data acquisition unit; wherein the data acquisition unit converts a received echo signal into a digital signal and then sends to the computer; wherein the computer provides beam formation of echo data, radio frequency signal envelope detection, and dynamic range compression of the digital signal received, for obtaining a laryngeal tissue structure image.

### 1) Overall Processes of the Preferred Embodiment

Referring to FIG. 1, a flow chart of the present invention is provided. An imaging and detecting method of vocal cord vibration based on PWU has two work modes. When an ultrasound linear array transducer is placed on a neck surface along a coronal section, a vocal cord vibration displacement image is able to be obtained with a displacement estimation algorithm, so as to quantitatively extract a vibration displacement of a vocal cord body layer, a false vocal cord vibration displacement, and an initial vocal cord displacement. When the ultrasound linear array transducer is placed on the neck surface along a cross section, UGGs of an entire vocal cord region and certain vocal cord tissue region are able to be obtained by calculating an echo signal intensity of a glottis, so as to quantitatively extract and measure feature points and phase parameters of vocal cord vibration.

### 2) Coronal Section Imaging of the Vocal Cords

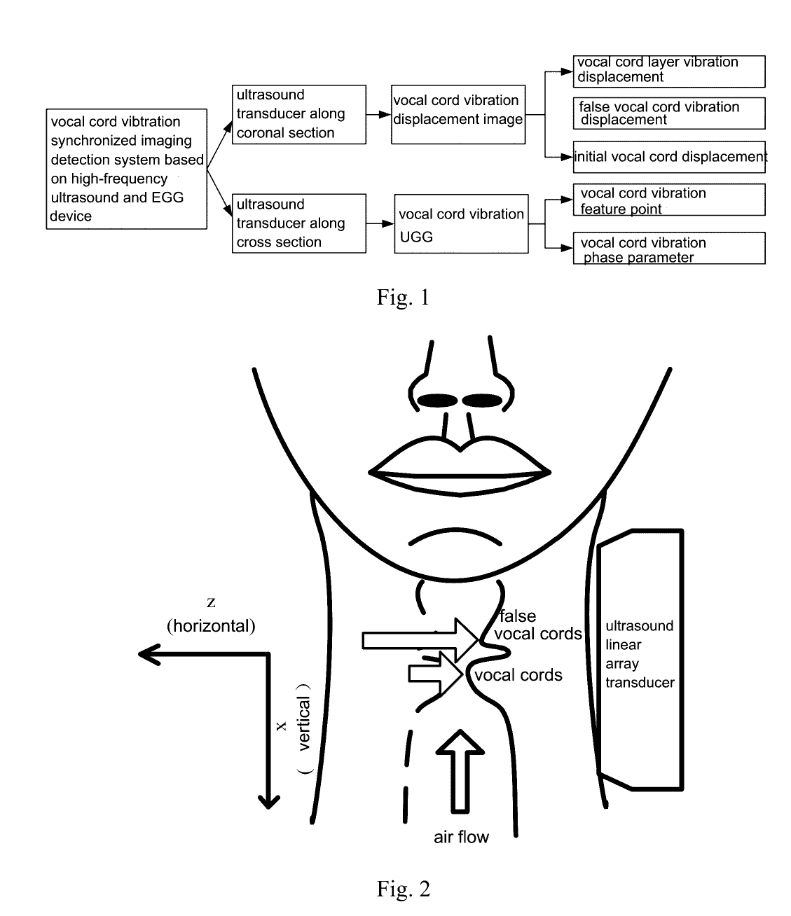

Referring to FIG. 2, a position of the ultrasound linear array transducer and a sectional view of the coronal section of a laryngeal tissue structure during the coronal section imaging of the vocal cords are provided, wherein a long arrow indicates a false vocal cord position and a short arrow indicates a vocal cord position. On a left side of FIG. 2, an x-z coordinate system is illustrated, wherein an x-axis indicates a vertical direction and a z-axis indicates a horizontal direction. The ultrasound linear array transducer is placed on a skin surface on a side of the neck where a laryngeal portion is. The vocal cords are small organs in a human body, which is below a thyroid cartilage. Therefore, in practice, the ultrasound linear array transducer with a center frequency of 7.2 MHz is used, in such a manner that an ultrasound signal passes through the thyroid cartilage while a sufficient image resolution is ensured. A vibration base frequency of the vocal cord has a range from dozens hertz to hundreds hertz. Therefore, for satisfying a Nyquist sampling theorem, an imaging frame rate is set to 5000 frames per second. Additionally, if the imaging frame rate is too high, the ultrasound linear array transducer will be too hot and thus be damaged, so no higher imaging frame rate is suggested.

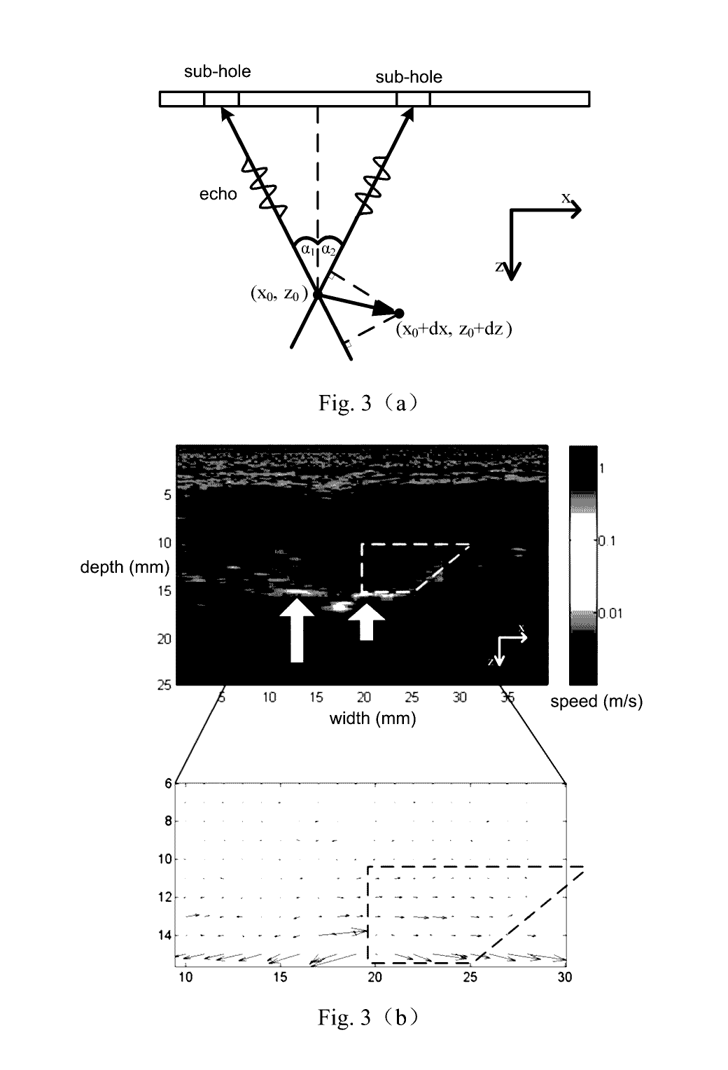

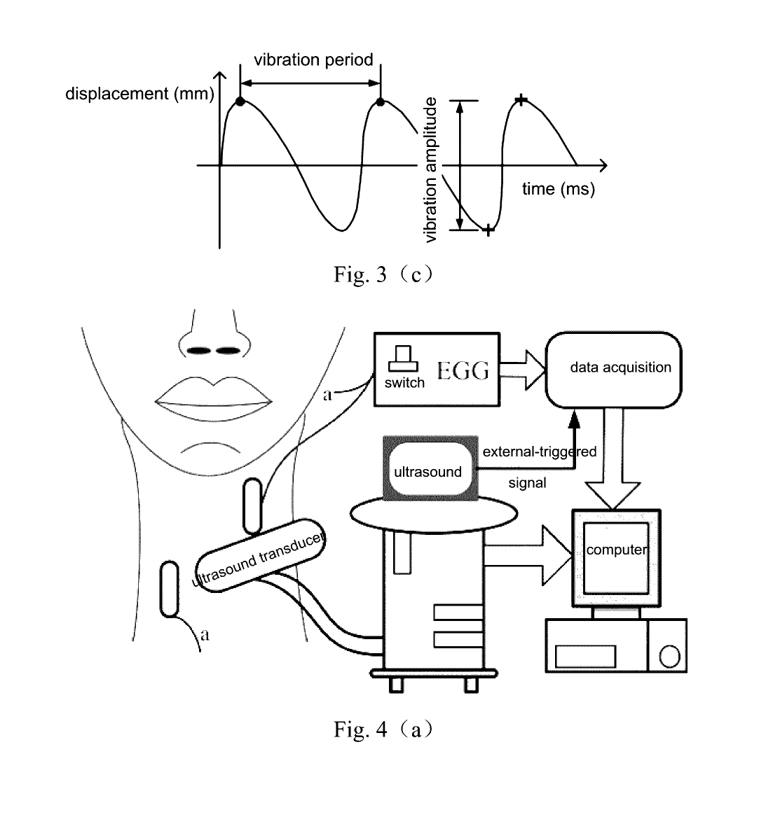

With stimulation of an ultrasound wave transmitter, the ultrasound linear array transducer sends a single pulse ultrasound plane wave with a width of 38 mm and a pulse period of 125 ns to a throat. The ultrasound plane wave will scatter after reaching tissues, and generate an echo with an opposite direction. The echo will be received by the ultrasound linear array transducer, so as to convert an echo signal into a digital signal by a multi-channel radio frequency data acquisition device and store the echo signal in a computer hard disk. After beam formation, radio frequency signal envelope detection, and dynamic range compression, echo data stored in the computer hard disk is converted into a laryngeal tissue structure image, as shown in FIG. 3(b). Referring to FIG. 3(b), a long arrow indicates a false vocal cord position and the short arrow indicates a vocal cord position. The ultrasound wave cannot pass through air between the vocal cords at both sides, so only the vocal cords on one side are observable in FIG. 3(b).

### 3) Tissue Vibration Measurement

The present invention is able to provide vocal cord vibration imaging while measure vibration speeds and displacements of the vocal cord tissues. Herein, a 2-dimensional motion estimation algorithm based on ultrasound radio frequency echo data is adopted.

FIG. 3(a) is a sketch view of the 2-dimensional motion estimation algorithm based on the ultrasound radio frequency echo data. An object of the algorithm is to measure a motion displacement and speed of a tissue at a position of (x₀, z₀) of FIG. 3(a). Supposing that at a next sampling time, the tissue at the position of (x₀, z₀) moves to a position of (x₀+dx, z₀+dz). Therefore, during a next sampling period, a tissue displacement is (dx, dz).

The algorithm comprises steps of: with a beam forming algorithm, obtaining tissue echo signals received from the position of (x₀, z₀) by two sub-holes of the transducer, and respectively marking as RF₁ and RF₂, wherein angles between the two sub-holes and a sound field axial direction are α₁ and α₂. Tissue movements cause delays of the RF₁ and the RF₂ and the delays are named as t_{α}_{₀} and t_{α}_{₁}. A relationship between the delays and the displacements is:

$${t}_{{α}_{0}} = \frac{dz + dz \cdot cos {α}_{0} + dx \cdot sin {α}_{0}}{c} , \\ {t}_{{α}_{1}} = \frac{dz + dz \cdot cos {α}_{1} + dx \cdot sin {α}_{1}}{c} . \qquad ( 1 )$$

wherein c represents a sound speed in the tissues. According to a 1-dimensional cross-correlation algorithm, the t_{α}_{₀} and the t_{α}_{₁} are calculated, so as to reversely calculate the tissue displacements:

$$dz = \frac{c \cdot {t}_{{α}_{0}} \cdot sin {α}_{1} + c \cdot {t}_{{α}_{1}} \cdot sin {α}_{0}}{( 1 + cos {α}_{0} ) \cdot sin {α}_{1} - ( 1 + cos {α}_{1} ) \cdot sin {α}_{0}} , \\ dx = \frac{c \cdot {t}_{{α}_{1}} \cdot ( 1 + cos {α}_{0} ) - c \cdot {t}_{{α}_{0}} \cdot ( 1 + cos {α}_{1} )}{( 1 + cos {α}_{0} ) \cdot sin {α}_{1} - ( 1 + cos {α}_{1} ) \cdot sin {α}_{0}} . \qquad ( 2 )$$

The imaging frame rate is known, so a sampling interval is available. With the algorithm, tissue displacements at all grid points within a visual range during the sampling interval are able to be calculated. By dividing the displacement with the sampling interval, an average tissue moving velocity during the sampling interval is calculated. Because the imaging frame rate is 5000 frames per second, the sampling interval is only 200 μs, which is much shorter than a vibration period of the vocal cord tissues. Therefore, the average tissue moving velocity approximately equals to a tissue instantaneous velocity. By processing the velocity with differential, a vibration displacement curve of the vocal cord tissues is obtained, as shown in FIG. 3(c). By detecting peak values and valley values of the curve, the vibration period of the vocal cords, the basic frequency of the vocal cords, and the vibration amplitude of the vocal cord tissues are able to be calculated.

### 4) Cross Section Imaging of the Vocal Cords

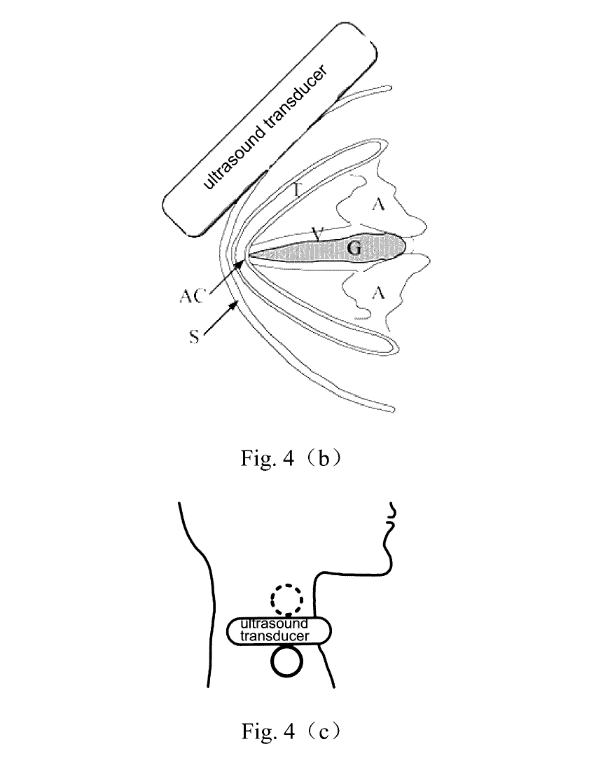

Firstly, the digital ultrasonography system is operated in a mode B which is conducive to clear imaging. After being coupled, the ultrasound linear array transducer is placed on the skin surface on the side of the testee neck along the cross section, which equals to a height of the glottis of the testee. Then the position and an angle of the ultrasound linear array transducer are finely adjusted until images of an anterior commissure and an arytenoid cartilage are simultaneously observable on a screen of the digital ultrasonography system. FIG. 4(b) is a sketch view of relative positions of the ultrasound transducer, the vocal cords and surrounding tissues thereof, wherein an outermost layer S represents skin, T represents the thyroid cartilage, and V represents the vocal cords. The vocal cords at both sides are combined into a vocal cord tendon attaching on the thyroid cartilage, which forms the anterior commissure (AC for short). An interval between the vocal cords on both sides is called a glottides rimae, or glottis (G for short). A rear portion A shows the arytenoid cartilage. When the anterior commissure and the arytenoid cartilage are simultaneously observable on the screen of the digital ultrasonography system, the entire vocal cords along a vocal cord length direction enters an imaging range.

When position of the anterior commissure and the arytenoid cartilage are found, a mode of the digital ultrasonography system is set to place wave imaging, wherein detailed imaging parameters are the same as that in above mentioned “Coronal section imaging of the vocal cords”, i.e. a single pulse ultrasound plane wave with a width of 38 mm and a pulse period of 125 ns is sent to the throat. An EGG electrode is placed above the transducer and on the neck surface, and another EGG electrode is placed bias-downwards at an opposite side of the neck. The two EGG electrodes are respectively 1 cm higher and lower than the glottis, as shown in FIGS. 4(b) and 4(c). Positions of the EGG electrodes should avoid a transmission path of an ultrasound beam, in case the ultrasound echo signal is interfered. While the testee pronounces, a tester pushes a record button of the digital ultrasonography system for recording the ultrasound RF data. Meanwhile, an external-triggered signal sent by the digital ultrasonography system controls an EGG device to simultaneously recording an EGG signal. An entire sampling process lasts for 250 ms, which usually covers dozens vocal cord vibration periods. All the RF data and the EGG data are stored in the computer and waits for follow-up off-line treatments.

### 5) Method for Extracting the UGG

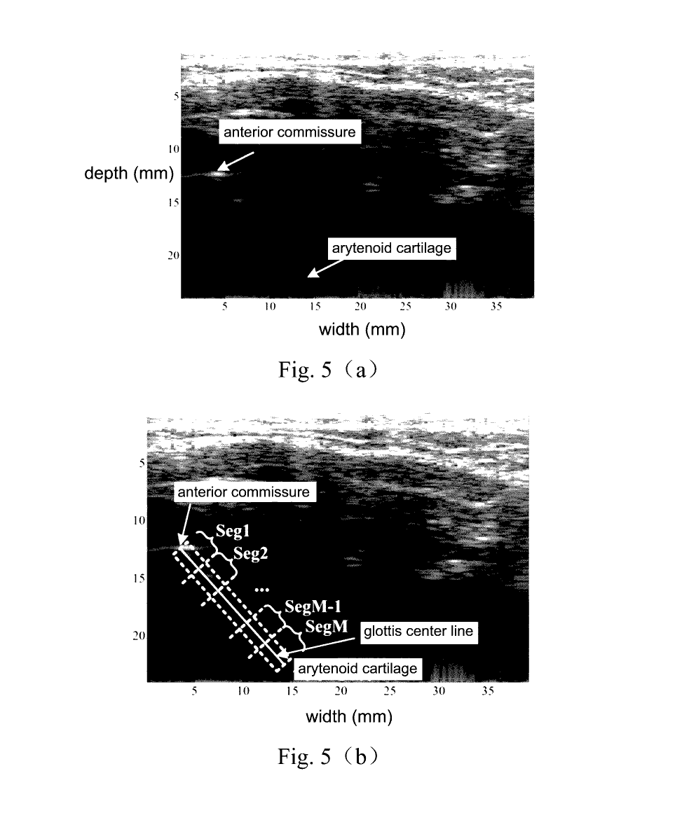

Referring to FIGS. 5(a) and 5(b), a frame of plane wave ultrasound image along a front-rear direction of the vocal cords is illustrated. When the vocal cords at both sides are separated by air flow from a lung and the glottis appears, an air-tissue interface will be formed at a vocal cord edge. The vocal cord vibration is periodical, so the glottis appears and disappears periodically. The air-tissue interface will strongly reflects the ultrasound signal, so strongly reflected echo signal, which appears and disappears periodically, is observable on the screen of the digital ultrasonography system, wherein in ultrasound image sequences, a bright line appears and disappears periodically. There are two bright areas at two ends of the bright line. The two bright areas exist in all the ultrasound image sequences with relatively fixed positions. The two bright areas are the positions of the anterior commissure and the arytenoid cartilage, as shown by arrows in FIG. 5(a). The bright line therebetween represents the echo signal of the air-tissue interface of the vocal cords. The PWU technologies are able to overcome space asynchronism of the conventional linear scan, so as to simultaneously obtain vocal cord vibration signals along the entire vocal cord length direction by measuring an ultrasound echo signal amplitude of a glottis area. A time-varying echo intensity curve measured is the UGG.

Firstly the positions of the anterior commissure and the arytenoid cartilage are subjectively judged, and the positions in the ultrasound image are manually connected with a line, wherein the line coincides with a central line of the glottis. Because of a changeable glottis shape and an ultrasound reverberation effect, the strong echo ultrasound signal of the air-tissue interface is illustrated as a line with a certain width in the ultrasound image. Therefore, a rectangle as region of interest (ROI for short) is selected, wherein a width of the ROI is 1-5 mm. The central line of the glottis coincides with a symmetry axis of the rectangle; then evenly dividing the ROI into a plurality of segments along a vocal cord length direction. Then gray values of all pixels in each of the segments of the ROI are extracted, for calculating a time-varying UGG of each of the segments of the ROI according to an equation (3):

$$U G G ( t ) = norm ( - \frac{1}{N} {\sum }_{i , j} {P}_{i , j} ( t ) ) \qquad ( 3 )$$

wherein, UGG(t) is the time-varying UGG, P_{i,j}(t) is a gray value of a pixel point (i,j) in the ROI at a time t; N is a quantity of all the pixels in the ROI; norm represents a normalizing operation; wherein the ROI is divided into M segments, and for each of the segments of the ROI, a corresponding UGG is extracted.

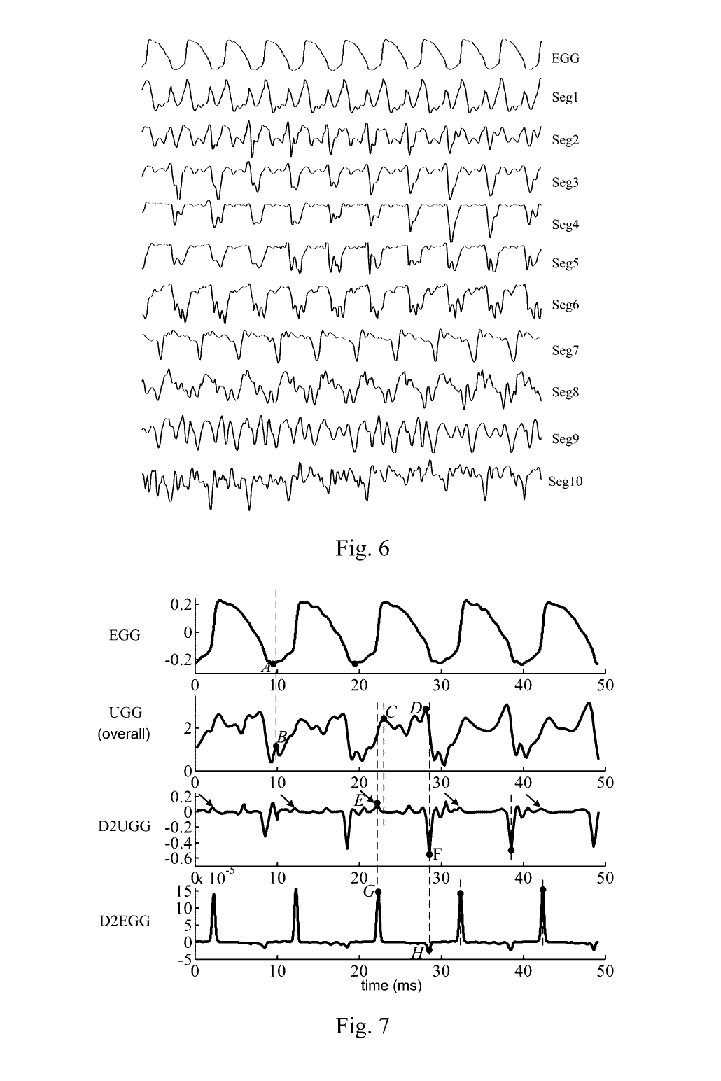

Referring to FIG. 6, the segmented UGG of 10 segments of the ROI of the vocal cords between the anterior commissure and the arytenoid cartilage is illustrated, wherein high-amplitude portions represent a weak ultrasound echo signal intensity, and low-amplitude portions represent a strong ultrasound echo signal intensity.

Because the positions of the anterior commissure and the arytenoid cartilage are subjectively judged, and the anterior commissure and the arytenoid cartilage have certain volumes, not all the extracted UGGs of the segments of the ROI reflect movement results of the vocal cords. When two sides of the vocal cords contact, the ultrasound wave beam is able to pass through the tissues contacted with the vocal cords; and when the two sides of the vocal cords are separated, most of the ultrasound wave beam is reflected by the air-tissue interface. Therefore, the UGG describing the vocal cord vibration should have large and small amplitudes appearing in certain regularity and order. Referring to FIG. 6, Seg 3, Seg, 4, Seg 5, Seg 6 and Seg 7 are five curves in the segmented UGG, which satisfy vocal cord vibration features on the ultrasound echo. By combining the curves, an overall UGG of the vocal cord vibration is able to be obtained, as shown in FIG. 7. Referring to FIG. 7, a synchronized EGG is also illustrated. By processing the EGG with differential, a DEGG is obtained, for calculating a D2EGG according to an equation 4). Similarly, by processing the UGG with differential, a DUGG is obtained, for calculating a D2UGG according to an equation (5).

D2EGG=DEGG(n)|DEGG(n)| (4)

D2UGG=DUGG(n)|DUGG(n)| (5)

### 6) Extraction of Feature Points and Feature Parameters of the UGG

Feature points of the EGG are capable of indicating very important phase times during the vocal cord vibration. According to a peak detection algorithm, a maximum glottis open time point A of the EGG, a glottis close time point G of the D2EGG, and a glottis open time point H are extracted. Meanwhile, corresponding feature points are extracted from the UGG. By adjusting a search window length of the peak detection algorithm, an echo intensity weakest point C in a glottis close phase and an echo intensity weakest point D in a glottis open phase of the overall UGG are obtained. A point B is a small but distinctive wave peak in each period of the overall UGG, wherein by finding a second zero-crossing point after the point D, the point B of each period is able to be extracted. A point E is a small positive peak of the D2UGG, which is extracted by extracting a first positive peak before the point C. A point F is a negative peak of the D2UGG, which is very distinctive and is easy to be detected by the peak detection algorithm.

In the EGG, the point A is a valley value point of the EGG, which represents maximum glottis open time. In the UGG, according to a vocal cord open phase, although amplitude of the overall UGG is relatively small, there is still a distinctive wave, and the marked point B is an apex of the wave peak. The point B represents a time after the vocal cords move towards both sides, when a reflected echo at the center line of the glottis is weakest. Therefore, a time of the point B is also the maximum glottis open time.

In the D2EGG, the point G is a positive peak value point of the D2EGG, which represents a time when the glottis is just closed. The point H is a negative value peak point of the D2EGG, which represents a time when the glottis is just opened. The E and F points in the D2UGG are respectively a positive peak point and a negative peak point, which also represent same vibration phase meanings.

a glottis closure quotient (CQ for short) represents a ratio of a vocal cord close period and an entire vocal cord vibration period. Generally, the CQ is simply extracted from the D2EGG according to an equation (6):

$$C Q = \frac{Loc ( H ) - Loc ( G )}{{T}_{egg}} \qquad ( 6 )$$

wherein Loc represents a time position of a point, and T_{egg} represents a vibration period length.

However, in many cases, the positive peak of the D2EGG is distinctive, while the negative peak is not so or even not identifiable. Nevertheless, the negative peak of the D2UGG is very distinctive. Therefore, by extracting the positive peak of the D2EGG and the negative peak of the D2UGG, a more reliable CQ is able to be calculated according to an equation (7):

$$C Q = \frac{Loc ( F ) - Loc ( G )}{{T}_{egg}} . \qquad ( 7 )$$

Therefore, by taking advantages of the EGG and the UGG, vocal cord vibration parameters obtained are more accurate and reliable.

One skilled in the art will understand that the embodiment of the present invention as shown in the drawings and described above is exemplary only and not intended to be limiting. It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An imaging and measuring system of vocal cord vibration based on plane wave ultrasonography, comprising: a digital ultrasonography system, a data acquisition unit, and a computer; wherein the digital ultrasonography system comprises an ultrasound linear array transducer and a host;
wherein the ultrasound linear array transducer is controlled by the host for sending an ultrasound plane wave and receiving an echo; the echo is sent back to the host;
wherein the host controls the ultrasound linear array transducer for sending the ultrasound plane wave, and sends back the echo to the data acquisition unit;
wherein the data acquisition unit converts a received echo signal into a digital signal and then sends to the computer;
wherein the computer provides beam formation of echo data, radio frequency signal envelope detection, and dynamic range compression of the digital signal received, for obtaining a laryngeal tissue structure image.

2. The imaging and measuring system, as recited in claim 1, wherein the ultrasound linear array transducer is placed on a neck surface of a testee along a coronal section, or along a cross section.

3. The imaging and measuring system, as recited in claim 1, wherein an imaging frame rate of the digital ultrasonography system is 5000 frames per second, a center frequency of the ultrasound linear array transducer is 7.2 MHz.

4. The imaging and measuring system, as recited in claim 1, wherein the ultrasound linear array transducer is placed on a neck surface of a testee along a coronal section; and the computer extracts vibration displacement of the vocal cord body layer, vibration displacement of the false vocal cord, and initial vocal cord displacement from the laryngeal tissue structure image with a 2-dimensional motion estimation algorithm based on ultrasound radio frequency echo data.

5. The imaging and measuring system, as recited in claim 1, wherein the ultrasound linear array transducer is placed on a neck surface of a testee along a cross section; and the computer extracts feature points and phase parameters of vocal cord vibration from the laryngeal tissue structure image.

6. A method for imaging of vocal cord vibration based on plane wave ultrasonography, comprising steps of:
placing ultrasound linear array transducers on a skin surface on a side of a testee neck, which is corresponding to a glottis position, along a coronal section and/or a cross section; sending an ultrasound plane wave to a laryngeal portion by the ultrasound linear array transducers, and receiving an echo, then sending the echo to a data acquisition unit;
converting a received echo signal into a digital signal by the data acquisition unit and then sending to a computer; providing beam formation of echo data, radio frequency signal envelope detection, and dynamic range compression of the digital signal received, by the computer, for obtaining a laryngeal tissue structure image.

7. A method for imaging of vocal cord vibration based on plane wave ultrasonography, comprising steps of: collecting a laryngeal tissue structure image by a computer, extracting vibration displacement of the vocal cord body layer, vibration displacement of the false vocal cord, and initial vocal cord displacement from a laryngeal tissue structure image with a 2-dimensional motion estimation algorithm based on ultrasound radio frequency echo data.

8. The method, as recited in claim 7, wherein the laryngeal tissue structure image is obtained by: placing an ultrasound linear array transducer on a skin surface on a side of a testee's neck, which is corresponding to a glottis position, along a coronal section; sending an ultrasound plane wave to a laryngeal portion by the ultrasound linear array transducers, and receiving an echo, then sending the echo to a data acquisition unit; converting a received echo signal into a digital signal by the data acquisition unit and then sending to the computer; providing beam formation of echo data, radio frequency signal envelope detection, and dynamic range compression of the digital signal received, by the computer.

9. A method for imaging of vocal cord vibration based on plane wave ultrasonography, comprising steps of: collecting an ultrasound glottograph (UGG for short), which is sampled by an ultrasound linear array transducer, by a computer; judging positions of an anterior commissure and an arytenoid cartilage, and connecting the positions in an ultrasound image with a line, wherein the line coincides with a central line of a glottis; then selecting a rectangle as region of interest (ROI for short), wherein the line which coincides with the central line of the glottis is a symmetry axis of the rectangle; then evenly dividing the ROI into a plurality of segments along a vocal cord length direction; extracting gray values of all pixels in each of the segments of the ROI, and calculating a time-varying UGG of each of the segments of the ROI according to an equation (3):
$$U G G ( t ) = norm ( - \frac{1}{N} {\sum }_{i , j} {P}_{i , j} ( t ) ) \qquad ( 3 )$$
wherein, UGG(t) is the time-varying UGG, P_{i,j}(t) is a gray value of a pixel point (i,j) in the ROI at a time t; N is a quantity of all the pixels in the ROI; norm represents a normalizing operation; wherein the ROI is divided into M segments, and for each of the segments of the ROI, a corresponding UGG is extracted;
then identifying curves with periodic alternating amplitudes from UGG waveforms which are extracted from each segmented ROI; combining the curves for obtaining an overall UGG of vocal cord vibration; processing the UGG with differential for obtain a differential ultrasound glottograph (DUGG for short), calculating a D2UGG according to an equation (5):
D2UGG=DUGG(n)|DUGG(n)| (5)
according to a peak detection algorithm, obtaining an echo intensity weakest point in a glottis close phase and an echo intensity weakest point in a glottis open phase of the overall UGG; wherein a maximum glottis open time point is a second zero-crossing point after the echo intensity weakest point in the glottis open phase of the overall UGG; a glottis close time point is a first positive peak before an echo intensity weakest point in a glottis close phase of the D2UGG; and a glottis open time point is a negative peak of the D2UGG;
calculating a glottis closure quotient (CQ for short) according to an equation (7):
$$C Q = \frac{Loc ( F ) - Loc ( G )}{{T}_{egg}} \qquad ( 7 )$$
wherein Loc(F) represents a time position of the negative peak of the D2UGG, Loc(G) represents a time position of a position peak of the D2UGG, and T_{egg} represents a vibration period length.

10. The method, as recited in claim 9, wherein the UGG is obtained by: placing the ultrasound linear array transducer on a skin surface on a side of a testee neck, which is corresponding to a glottis position, along a cross section; sending an ultrasound plane wave to a laryngeal portion by the ultrasound linear array transducers, and receiving an echo, then sending the echo to a data acquisition unit; converting a received echo signal into a digital signal by the data acquisition unit and then sending to the computer, for obtaining a curve of the time-varying echo intensity.
